(19) United States
(12) Patent Application Publication
(10) Pub. No.: US 2004/0162350 A1
(43) Pub. Date: Aug. 19, 2004

(54) Method of treating a non-cancerous disease of the male prostate

(76) Inventor: Arthur Vanmoor, Boca Raton, FL (US)

Correspondence Address: OTTO S. KAUDER, 5382C Venetia Ct., Boynton Beach, FL, 33437, US

(21) Appl. No.: 10/368,218

(22) Filed: Feb. 18, 2003

(51) Int. Cl. A61K 31/198

(52) U.S. Cl. 514/561; 514/563

(57) ABSTRACT

There is disclosed a method of treating a non-cancerous disease condition of the male prostate gland in a person in need of such treatment, which comprises the administration to such person of an amount of water-soluble aminoacid represented by formula (I)

X—CₙH₂ₙ—CR(NH₂)COOH (I)

wherein X represents a hydrogen atom (H) or a carbamoyl group (CONH₂), R is hydrogen or a methyl (CH₃) group, and n is a whole number from 1 to 4, effective in diminishing the said of said prostate gland. The disease condition can be any one or more of non-cancerous enlargement of the prostate gland, non-cancerous infection of the prostate gland, and non-cancerous inflammation of the prostate gland.

### BACKGROUND OF THE INVENTION

1. Field of the Invention

This invention relates to a method of treating a male person suffering from a non-cancerous disease condition of the prostate gland with an agent effective in counteracting that condition and minimizing associated pain and discomfort.

2. Description of the Related Art

As is well known, cancerous conditions are characterized by the uncontrolled growth of tissues commonly known as tumors. Traditional attempts to mitigate the unfavorable effects of these have included surgical removal of tumors, administration of drugs that result in diminution of tumors, treatment with ionizing radiation to kill tumors, and all the possible combinations of these. The MERCK MANUAL, 16^{th} edition, published 1992, at pages 1287 to 1292, which portion is here incorporated by reference, contains a table titled “Five Year Disease-free Survival Rates for Cancers Treated with Combined Therapies” listing the effectiveness in percent of various combination treatments against particular cancers at various stages. The table shows for only one out of 18 types of cancer a five year survival rate as high as 90% and the subsequent discussion confirms that known therapies are limited in their effectiveness and accompanied by well known harmful side effects of each.

As is also well known, the search for better remedies for this as well as other suffering conditions is enormously costly. For economic reasons, moreover, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in U.S. Pat. No. 5,616,617 as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are disclosed in U.S. Pat. No. 5,767,157 as effective against pain in a joint.

For evident reasons, however, this method is not applicable to the study and relief of cancerous conditions.

One type of cancer condition that lends itself well to monitoring in simple ways without the need for hospitalization or the use of complex equipment is cancer of the prostate in the human male. It develops slowly over many years and is not considered highly life threatening. Yet even in its early stages it manifests itself in the well known enlargement of the prostate gland that results in pressure on the bladder and consequently a need for the victim to urinate much more frequently than a person without this condition, thus signaling a need to determine by appropriate test whether a cancer condition exists.

However, prostate cancer is not the only disease condition that can affect the male prostate gland. The prostate is the most common site of disorders in the male genitourinary system, and there also exist such non-cancerous problems of the male prostate gland as prostatitis, which is inflammation of the prostate, and benign prostatic hypertropohy or BPH. Prostatitis can be infectious, resultling from bacterial invasion of the gland itself or from attack on the bladder or the kidney, and can be acute or chronic. There are also non-infectious forms of prostatitis whose cause is not known.

Distinction of prostate cancer from BPH is not a simple matter. For example, P. A. Balch CNC and J. F. Balch MD in the book “Prescription for Nujtritional Healing” (New York: Avery Publishing Group, 3^{rd} edition, 2000) devote pages 591-596 to a discussion of prostate cancer and pages 596-598 to a discussion of prostatitis (an inflammatory condition) and BPH. These pages are here incorporated by references. The authors illustrate the difficulty of distinguishing between the conditions by describing a “blood test to detect elevated levels of a protein called prostate-specific antigen (PSA)” as an excellent screening test for prostate cancer (page 592, left) but within the same paragraph also state, “high PSA levels can be caused by factors other than cancer, including benign enlargement or inflammation of the prostate, an activity as innocuous as bicycle riding, or even the rectal exam itself.”

“Ultimately,” as the authors note in the following paragraph, “a tissue diagnosis must be done” to confirm a cancerous condition; this can be done only by an invasive procedure such as microscopic examination of a needle biopsy.

There clearly remains a need for new approaches to finding and applying more effective and also less aggressive techniques for distinguishing a non-cancerous enlargement of the male prostate gland from a cancerous condition, and for treating such a non-cancerous enlargement in a non-aggressive manner.

### SUMMARY OF THE INVENTION

In accordance with this invention, there is provided a method of treating a non-cancerous disease of the prostate in a male person in need of such treatment, which is enlargement of the prostate and which comprises the administration to such person of an amount of a water-soluble aminoacid represented by formula (I) shown below, effective in mitigating that disease.

Also in accordance with this invention, there is provided a method of treating a non-cancerous disease of the prostate in a male person in need of such treatment, which is infection of the prostate and which comprises the administration to such person of an amount of a water-soluble aminoacid represented by formula (I) shown below, effective in mitigating that disease.

Also in accordance with this invention, there is provided a method of treating a non-cancerous disease of the prostate in a male person in need of such treatment, which is inflammation of the prostate and which comprises the administration to such person of an amount of a water-soluble aminoacid represented by formula (I) shown below, effective in mitigating that disease.

X—CₙH₂ₙ—CR(NH₂)COOH (I)

In formula (I), X represents a hydrogen atom (H) or a carbamoyl group (CONH₂), R is hydrogen or a methyl (CH₃) group, and n is a whole number from 1 to 4. When n is greater than 1, the alkylene group represented by CₙH₂ₙ can be straight chain or branched.

The effectiveness of the aminoacid represented by formula (I) according to the invention is believed to accompany enhancement of the effectiveness of the person's immune system.

The present invention is based on the recognition that enhancing the effectiveness of the immune system in a person can be beneficial in augmenting the person's innate ability to eliminate toxins from the body and to resist the initiation of the process that leads to a condition of non-cancerous disease of the prostate gland as well as to slow down, arrest, and even reverse that process. As a result, the incidence of painful urination, sleep deprivation, and other discomfort associated with such disease is diminished, and the quality of life is improved.

In minimizing non-cancerous disease of the male prostate gland according to this invention, mega-nutrient doses of 2 to 20 grams of a water-soluble aminoacid represented by formula (I) can be administered from one to five times daily, for a total of 2 to 100 grams per day, until monitoring shows sufficient improvement in the user's condition to permit reduction in dose level and ultimately cessation of the treatment. Daily doses of a water-soluble aminoacid represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices. Alternatively, there can be given sterile solutions of a water-soluble aminoacid represented by formula (I) by direct injection into the bloodstream of the person to be treated, as well as by rectal suppositories containing a water-soluble aminoacid represented by formula (I).

In formula (I), when n=1 the alkylene group represented by CₙH₂ₙ is a methylene group. When n=2, the alkylene group can be ethylene —CH₂CH₂— or ethylidene —CH(CH₃)—. When n=3 and n=4, the alkylene group represented by CnH₂ₙ can have each of the known isomeric configurations of such straight chain and branched alkylene groups.

The following table contains preferred water-soluble amino acids represented by formula (I) according to the invention.

| Index | Name | X | R | n |
|---|---|---|---|---|
| 1 | 2-aminobutanoic acid | H | H | 2 |
| 2 | 2-amino-2-methylpropanoic acid | H | CH₃ | 1 |
| 3 | 2-amino-3-carbamoylpropanoic acid | CONH₂ | H | 1 |
| 4 | 2-amino-4-carbamoylbutanoic acid | CONH₂ | H | 2 |
| 5 | 2-amino-3-methylpentanoic acid | H | H | 4 |
| 6 | 2-amino-4-methylpentanoic acid | H | H | 4 |
| 7 | 2-amino-3-methylbutanoic acid | H | H | 3 |

### EXAMPLE 1

A group of male volunteers recorded for six weeks the number of times a night that they were awakened with the need to urinate. Those who woke up three or more times a night in more than 28 nights durng the six week trial were then given multiple daily doses of 15-20 grams of 2-amino-4-carbamoylbutanoic acid and continued to record the number of times a night that they were awakened with the need to urinate.

Over time, there were volunteers whose awakening at night with the need to urinate diminished from three or more times a night to twice, once, and even none at all in at least two thirds of the nights in a subsequent six week trial period.

### EAMPLE 2

A male with prostate enlargement diagnosed as a non-cancerous condition started a regimen of taking 10 grams of 2-amino-4-carbamoylbutanoic acid with each meal for a total of 40 grams daily. He felt relief after a few weeks; after two months of this regimen the prostate was found to have shrunk to normal size.

What is claimed is:

1. A method of treating a non-cancerous disease of the prostate in a male person in need of such treatment, which is enlargement of the prostate and which comprises the administration to such person of an amount of water-soluble amino acids represented by formula (I)
X—CₙH₂ₙ—CR(NH₂)COOH (I)
wherein X represents a hydrogen atom (H) or a carbamoyl group (CONH₂), R is hydrogen or a methyl (CH₃) group, and n is a whole number from 1 to 4, effective in diminishing the size of said person's prostate.

2. A method of treating a non-cancerous disease of the prostate in a male person in need of such treatment, which is infection of the prostate and which comprises the administration to such person of an amount of water-soluble amino acids represented by formula (I)
X—CₙH₂ₙ—CR(NH₂)COOH (I)
wherein X represents a hydrogen atom (H) or a carbamoyl group (CONH₂), R is hydrogen or a methyl (CH₃) group, and n is a whole number from 1 to 4, effective in diminishing infection of said person's prostate.

3. A method of treating a non-cancerous disease of the prostate in a male person in need of such treatment, which is inflammation of the prostate and which comprises the administration to such person of an amount of water-soluble amino acids represented by formula (I)
X—CₙH₂ₙ—CR(NH₂)COOH (I)
wherein X represents a hydrogen atom (H) or a carbamoyl group (CONH₂), R is hydrogen or a methyl (CH₃) group, and n is a whole number from 1 to 4, effective in diminishing inflammation of said person's prostate.

4 The method of claim 1, wherein the aminoacid is administered orally with food.

5. The method of claim 1, wherein the aminoacid is administered by injection into the bloodstream.

6. The method of claim 1, wherein the aminoacid is administered by rectal suppository.

7. The method of claim 1, wherein the effective amount of the aminoacid is administered in one to five daily doses, each dose being in the range of 2 to 20 gram.

8. The method of claim 1, wherein the total of said effective amount of the aminoacid administered daily is in the range of 2 to 100 grams.

9. The method of claim 8, wherein the total of said effective amount of the aminoacid administered daily is in the range of 20 to 50 grams.

10. The method of claim 1, wherein said person experiences relief from the effects of said condition.

11. The method of claim 1 wherein after treatment said condition is not detectable.

12. The method of claim 1 wherein the aminoacid is 2-amino-4-carbamoylbutanoic acid.

13. The method of claim 2 wherein the aminoacid is 2-amino-4-carbamoylbutanoic acid.

14. The method of claim 3 wherein the aminoacid is 2-amino-4-carbamoylbutanoic acid.

15. The method of claim 2, wherein the effective amount of the aminoacid is administered in one to five daily doses, each dose being in the range of 2 to 20 gram.

16 The method of claim 2, wherein the total of said effective amount of the aminoacid administered daily is in the range of 2 to 100 grams.

17. The method of claim 3 wherein the effective amount of the aminoacid is administered in one to five daily doses, each dose being in the range of 2 to 20 gram.

18. The method of claim 3, wherein the total of said effective amount of the aminoacid administered daily is in the range of 2 to 100 grams.
